(19) United States
(12) Patent Application Publication
(10) Pub. No.: US 2012/0209176 A1
(43) Pub. Date: Aug. 16, 2012

(54) BALLOON CATHETER

(75) Inventor: JAMES M. ANDERSON, FRIDLEY, MN (US)

(73) Assignee: BOSTON SCIENTIFIC SCIMED, INC., MAPLE GROVE, MN (US)

(21) Appl. No.: 13/369,984

(22) Filed: Feb. 9, 2012

Related U.S. Application Data
(60) Provisional application No. 61/441,155, filed on Feb. 9, 2011.

(51) Int. Cl. A61M 25/10; A61L 29/16

(52) U.S. Cl. 604/103.02; 604/103.06

(57) ABSTRACT

Medical devices and methods for making and using the same are disclosed. An example medical device may include a fixed wire balloon catheter. An example fixed wire balloon catheter may include a core wire. An inner tubular member may be attached to the core wire. The inner tubular member may have a plurality of slots formed therein. A catheter shaft may be disposed about the inner tubular member. An inflation lumen may be defined between the catheter shaft and the core wire. A balloon may be coupled to the catheter shaft. The balloon may have a distal portion. A crossing tip may be coupled to the distal portion of the balloon.

### CROSS-REFERENCE TO RELATED APPLICATION

This application claims the benefit of U.S. Provisional Application Ser. No. 61/441,155, filed Feb. 9, 2011, the entire disclosure of which is incorporated herein by reference.

### FIELD OF THE INVENTION

The present invention pertains to medical devices, and methods for manufacturing medical devices. More particularly, the present invention pertains to fixed wire balloon catheters.

### BACKGROUND

A wide variety of intracorporal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

Embodiments of the present disclosure provide design, material, manufacturing method, and use alternatives for medical devices and tubular members for use in medical devices. An example medical device may include a fixed wire balloon catheter. An example fixed wire balloon catheter may include a core wire. An inner tubular member may be attached to the core wire. The inner tubular member may have a plurality of slots formed therein. A catheter shaft may be disposed about the inner tubular member. An inflation lumen may be defined between the catheter shaft and the core wire. A balloon may be coupled to the catheter shaft. The balloon may have a distal portion. A crossing tip may be coupled to the distal portion of the balloon.

Another example fixed wire balloon catheter may include a core member. A tubular member may be disposed about the core member. The tubular member may have a distal portion and may have an inflation lumen defined therein. The tubular member may have a plurality of slots formed therein. A balloon may be coupled to the distal portion of the tubular member. The balloon may have a distal waist. A sealing member may be coupled to the tubular member. The sealing tube may contact at least one of an inner surface and an outer surface of the tubular member. The sealing member may form a fluid tight seal that prevents fluid from passing through the slots formed in the tubular member. A crossing tip may be coupled to the distal waist of the balloon. Another example fixed wire balloon catheter may include a catheter shaft having a length. A balloon may be coupled to the catheter shaft. A torque assembly may extend through the catheter shaft. The torque assembly may include a core member, a tubular member attached to the core member, and a crossing tip attached to the tubular member. The tubular member may have a plurality of slots formed therein. The torque assembly may have a length that is at least as long as the length of the catheter shaft. The torque assembly may be rotatable relative to the catheter shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The devices and methods of the present disclosure may be more completely understood in consideration of the following detailed description of various embodiments in connection with the accompanying drawings, in which:

FIG. 1 is a partially cross-sectional side view of an example medical device;

FIG. 2 is a perspective view of an example tubular member;

FIG. 3 is a partially cross-sectional side view of a portion of the example medical device shown in FIG. 1;

FIG. 4 is a perspective view of an example crossing tip;

FIG. 5 is a perspective view of another example crossing tip;

FIG. 6 is a partially cross-sectional side view of another example medical device;

FIG. 7 is a partially cross-sectional side view of another example medical device; and

FIG. 8 is a partially cross-sectional side view of another example medical device.

While the embodiments described herein are amenable to various modifications and alternative forms, specifics thereof have been shown by way of example in the drawings and will be described in detail. It should be understood, however, that the intention is not to limit the devices and methods to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

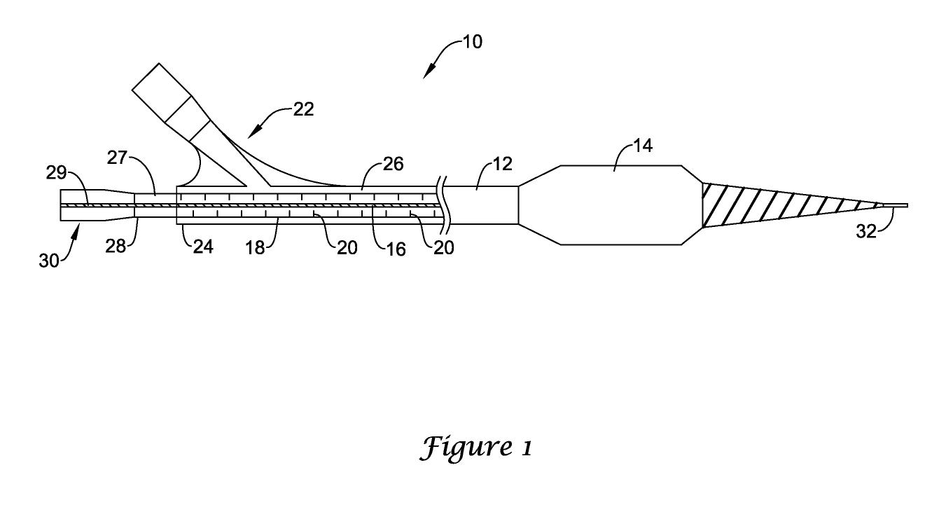

FIG. 1 is a partially cross-sectional side view of an example medical device 10 that, in this example, takes the form of a catheter. In at least some embodiments, catheter 10 may be a fixed wire catheter shaft. Fixed wire catheters differ from other so-called “over-the-wire” or “single-operator-exchange” catheters in a number of ways. For example, fixed wire catheters are navigated through the anatomy without the use of a guidewire (e.g., fixed wire catheters typically do not track along a guidewire). Thus, fixed wire catheter shaft (including, for example, catheter 10) may lack a hollow central lumen or guidewire lumen. In some instances, this may be desirable. For example, because catheter 10 may lack a guidewire lumen, it may be possible to manufacture a fixed wire catheter with a lower profile that catheters that include guidewire lumens. This may allow fixed wire catheters (including, for example, catheter 10) to access portions of the anatomy that might otherwise prove challenging to access. Fixed wire catheters (including, for example, catheter 10) may also provide a number of other additional desirable benefits and features.

Catheter 10 may include a catheter shaft 12. An expandable member or balloon 14 may be coupled to catheter shaft 12. In at least some embodiments, balloon 14 may be a drug eluting balloon. Other structures and configurations are also contemplated for balloon 14. A core wire or member 16 may be disposed within catheter shaft 12. In at least some embodiments, core wire 16 may take the form of or otherwise function as a steering or torque-transmitting member that can be used to efficiently transmit torque along the length of catheter shaft 12. Thus, core wire 16 may aid in steering and/or positioning of catheter shaft 12 (and/or balloon 14) during the advancement of catheter 10 through the anatomy. In some embodiments, core wire 16 takes the form of a wire and, thus, is solid in cross-section. Alternatively, core wire 16 may be tubular.

Catheter 10 may also include a tubular member 18. Tubular member 18 may be attached to or otherwise coupled with core wire 16. For example, core wire 16 may be attached to tubular member 18 along an inner surface of tubular member 18. Alternatively, core wire 16 may be attached to tubular member 18 along an outer surface of tubular member 18. The manner in which core wire 16 is attached to tubular member 18 may vary. In some embodiments, core wire 16 may be attached to tubular member 18 by welding, brazing, with an adhesive bond, with a mechanical bond, combinations thereof, or the like, or in any other suitable manner.

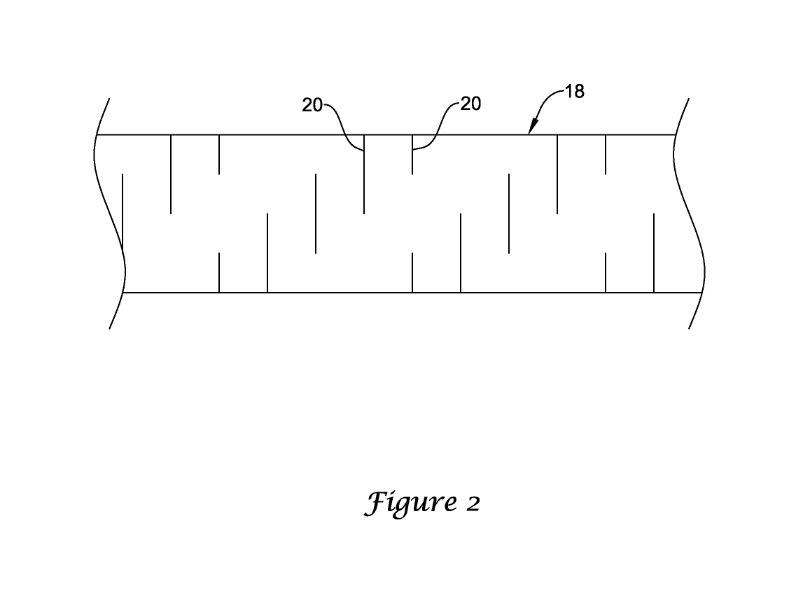

Tubular member 18 may have a plurality of slots 20 formed therein. Slots 20 may vary in configuration, number, arrangement, etc. For example, in some embodiments slots 20 may be disposed at an angle relative to the longitudinal axis of tubular member 18. In some of these and in other embodiments, slots 20 may lie within a plane that is substantially normal to the longitudinal axis of tubular member 20, for example, as illustrated in FIG. 2. These are just examples. Some additional details regarding some of the other configurations contemplated can be found below. It can be appreciated that any of the slot configurations and/or arrangements disclosed herein may be utilized, to the extent applicable, in any of the catheters and/or tubular members disclosed herein.

A hub or side port 22 may be provided at or adjacent to a proximal end 24 of catheter shaft 12. Port 22 may provide access to an inflation lumen 26 formed within catheter shaft 12 that is in fluid communication with balloon 14. In general, inflation lumen 26 may be defined between the inner surface of catheter shaft 12 and the outer surface of core wire 16. Inflation lumen 26 may or may not include portions or all of the interior of tubular member 18. Thus, inflation media may or may not pass through slots 20 during inflation of balloon 14.

Tubular member 18 may have a proximal end 28. In at least some embodiments, proximal end 28 may extend back to at least proximal end 24 of catheter shaft 12. In some of these and in other embodiments, proximal end 28 of tubular member 18 may extend proximally from proximal end 24 of catheter shaft 12. A proximal portion 27, which may extend proximally from proximal end 24 of catheter shaft 12 may lack slots. However, this is not required. Other variations are contemplated where proximal portion 27 includes slots. Some of these embodiments may include a sleeve (not shown), which may be disposed along the exterior of tubular member 18 and seal proximal portion 27.

A proximal end 29 of core wire 16 may extend to proximal end 28 of tubular member 18 or proximally therefrom. Regardless of the arrangement of proximal end 28 of tubular member 18 relative to proximal end 24 of catheter shaft 12, proximal end 28 of tubular member 18 (and/or proximal end 29 of core wire 16) may be attached to a torque member 30, which is accessible to a clinician using catheter 10. Torque member 30 may be used to apply torque to tubular member 18 and/or core wire 16, which can be transmitted along the length of core wire 16. Accordingly, rotation of torque member 30 may result in substantially equivalent rotation at a distal end 32 of core wire 16. This may desirably allow for reliable and predictable rotation of balloon 14.

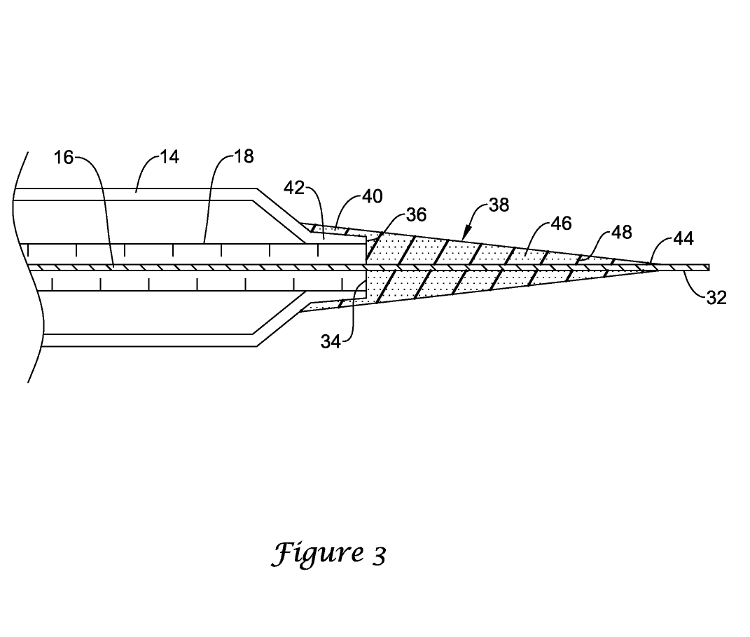

At the distal end of catheter 10, which is shown in FIG. 3, a distal end 34 of tubular member 18 may extend to a distal end 36 of balloon 14. Distal end 34 of tubular member 18 may be closed or otherwise sealed. This may allow, for example, inflation lumen 26 to be sealed at distal end 36 of balloon 14. Distal end 32 of core wire 16 may extend distally beyond distal end 36 of balloon 14. Distal end 32 of core wire 16 may also extend distally beyond distal end 34 of tubular member 18. Because of this, it can be appreciated that core wire 16 may have a length that is at least as great (or greater) as the length of tubular member 18 and/or at least as great (or greater) as the length of the catheter shaft 12.

A crossing tip member 38 may be attached to core wire 16. Crossing tip member 38 may be used, for example, to aid in passing catheter 10 through a vascular occlusion. The occlusion may include a chronic total occlusion, a fibrous occlusion, a calcified occlusion or lesion, and/or the like. In general, crossing tip member 38 may have a tapered or conical configuration so as to make it easier for tip member 38 to enter into an occlusion. In addition, because crossing tip member 38 is attached to core wire 16 (e.g., distal end 32 of core wire 16 may extend to or distally beyond a distal end 44 of crossing tip member 38), a user may be able to apply torque to torque member 30 in order to effect rotation of crossing tip member 38. Thus, crossing tip member 38 may be used in an auger-like manner to bore into and ultimately cross the occlusion. Numerous methods are contemplated for using crossing tip member 38 through an occlusion.

Crossing tip member 38 may also be attached to balloon 14. For example, a proximal portion or end 40 of crossing tip member 38 may be bonded to a distal waist 42 of balloon 14. Thus, crossing tip member 38 may be described as overlapping with balloon 14. Alternatively, crossing tip member 38 may be disposed at (e.g., abut) or disposed just distally of distal waist 42. Numerous configuration are contemplated.

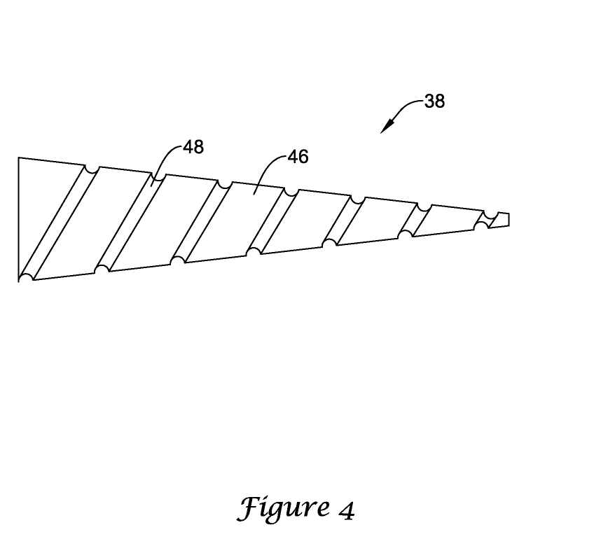

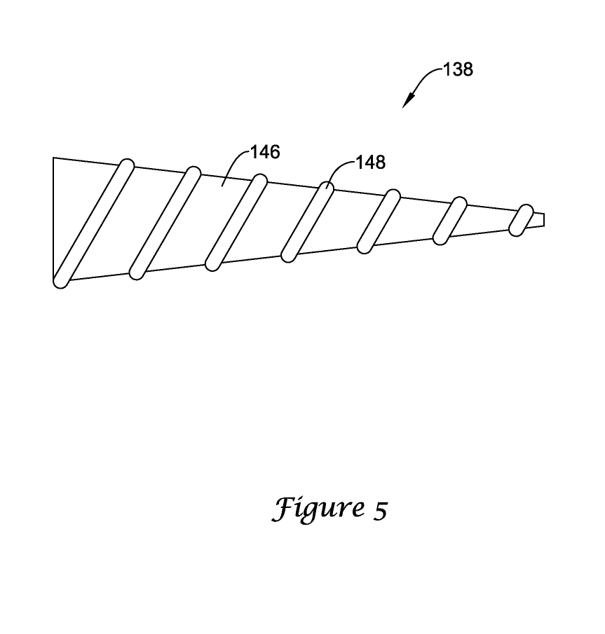

Crossing tip member 38 may have an uneven outer surface 46 that may further enhance the ability of catheter 10 to pass an occlusion. For example, outer surface 46 may include one or more grooves 48 formed therein as illustrated in FIG. 4. In at least some embodiments, groove 48 may include a helical groove. This, however, is not intended to be limiting as many different groove configurations are contemplated. Indeed, different outer surfaces are contemplated. For example, FIG. 5 illustrates outer surface 146 of crossing tip member 138, which may otherwise be similar in form and function to tip member 38, that includes one or more projections or threads 148. In at least some embodiments, thread 148 may including a helical thread. This, however, is not intended to be limiting as many different thread configurations are contemplated. For example, crossing tip members are contemplated that may include an outer cutting thread having a pointed (e.g., triangular) or sharpened outer surface so as to improve crossing, an exterior round or ribbon coil, other exterior surface features, combinations thereof, or the like.

Crossing tip member 38, and/or other crossing tip members disclosed herein, may generally include polymer materials. Some examples of suitable materials are disclosed herein. In some embodiments, crossing tip member 38 may include a hardened plastic material or a metal-polymer composite material. Alternatively, crossing tip member 38 may include a metal material or any other suitable material including those disclosed herein.

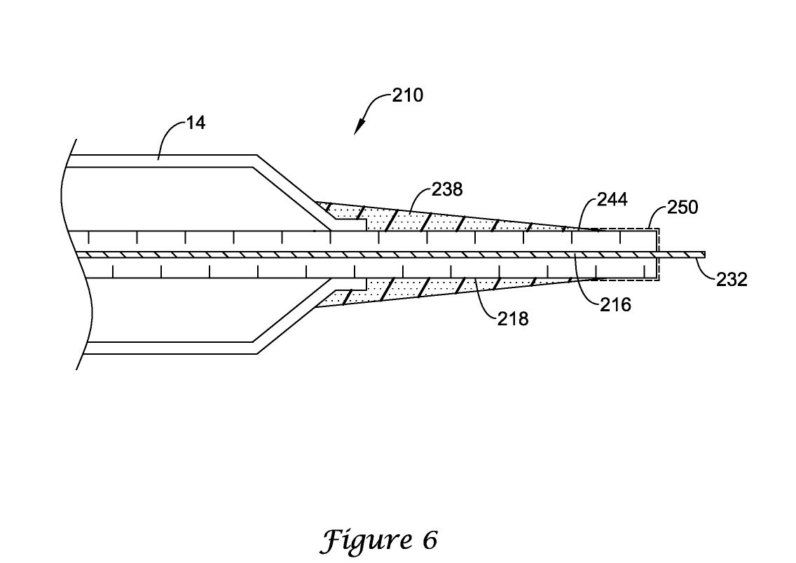

FIG. 6 illustrates a portion of another example catheter 210, which may be similar in form and function to other catheters disclosed herein. In this embodiment, tubular member 218 may extend distally beyond distal end 244 of crossing tip member 238. Distal end 232 of core wire 216 may extend distally beyond distal end 244 of crossing tip member 238 and may extend distally beyond distal end 234 of tubular member 218. In at least some embodiments, a sleeve or jacket 250 may seal the portion of tubular member 218 extending distally beyond distal end 244 of crossing tip member 238 and/or may seal distal end 234 of tubular member 218. Other embodiments are contemplated where a different structures or different arrangements may be utilized to seal tubular member 218. For example, some embodiments of tubular member 218 may lack slots at positions distal of balloon 214 as to maintain a fluid tight seal at the distal end of catheter 210.

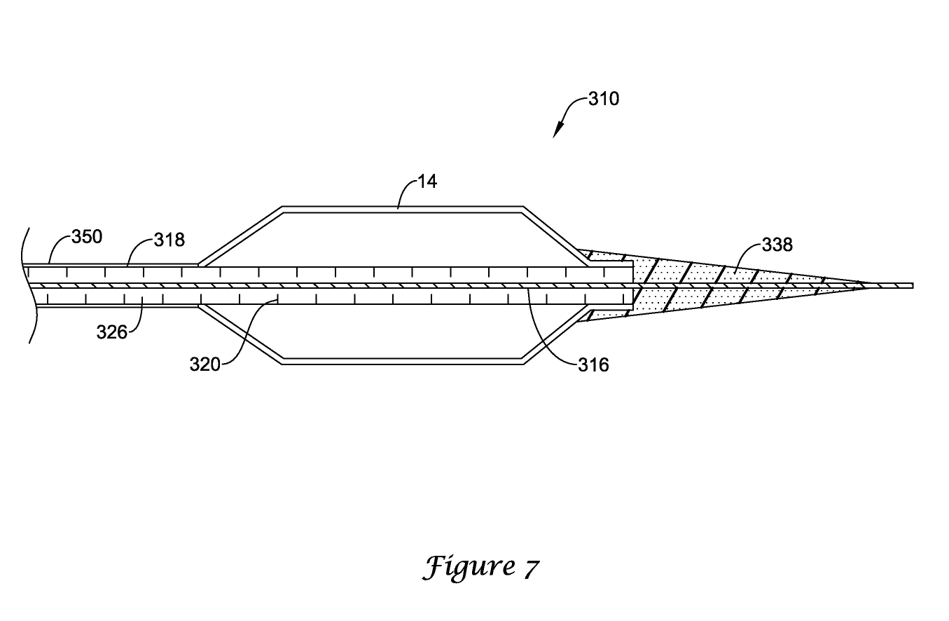

FIG. 7 illustrates another example catheter 310, which may be similar in form and function to other catheters disclosed herein. In this embodiment, tubular member 318 forms the catheter shaft and defines inflation lumen 326. Thus, catheter 310 may lack a catheter shaft that is distinct from tubular member 318. Sleeve 350 may be disposed along, for example, the exterior of tubular member 318 and seal slots 320 in tubular member 318. Catheter 310 may also include crossing tip member 338 and core wire 316.

Inflation of balloon 314 may include passing inflation media through tubular member 318. At positions under balloon 314, the inflation media may flow through slots 320 and into balloon 314. Alternatively, one or more inflation openings may be formed in tubular member 318 adjacent to (e.g., under) balloon 314 so as to allow for balloon 314 to be inflated.

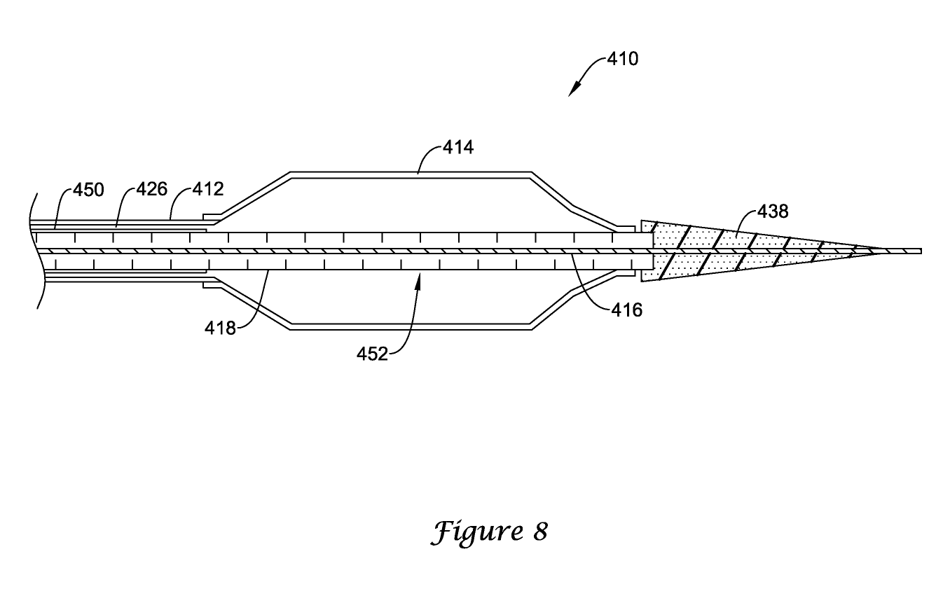

FIG. 8 illustrates another example catheter 410, which may be similar in form and function to other catheters disclosed herein. Catheter 410 may include a torque assembly 452, which may include tubular member 418, core wire 416, and crossing tip member 438. Unlike at least some of the other crossing tip members disclosed herein, crossing tip member 438 is free from attachment to balloon 414. This may allow torque assembly 452 to be rotatable relative to balloon 414. Accordingly, torque assembly 452 may be rotated so as to improve the ability of catheter 410 to pass an occlusion. For example, torque assembly 452 may be rotated in an auger-like manner to bore through the occlusion.

Catheter shaft 412 may define inflation lumen 426. For example, inflation lumen 426 may be defined between an interior wall surface of catheter shaft 412 and an exterior wall of sleeve 450. In some embodiments, sleeve 450 is an interior wall surface of catheter shaft 412. In other embodiments, sleeve 450 is a jacket disposed along tubular member 418 so as to form a structural interface between catheter shaft 412 and tubular member 418. Either way, sleeve 450 is configured to allow torque assembly 452 to rotate relative to catheter shaft 412 (and balloon 414) in the manner disclosed herein.

Various embodiments of arrangements and configurations of slots 20 are contemplated that may be used in addition to what is described above or may be used in alternate embodiments. For example, in some embodiments, at least some, if not all of slots 20 are disposed at the same or a similar angle with respect to the longitudinal axis of tubular member 18. For example, slots 20 can be disposed at an angle that is perpendicular, or substantially perpendicular, and/or can be characterized as being disposed in a plane that is normal to the longitudinal axis of tubular member 18. However, in other embodiments, slots 20 can be disposed at an angle that is not perpendicular, and/or can be characterized as being disposed in a plane that is not normal to the longitudinal axis of tubular member 18. Additionally, a group of one or more slots 20 may be disposed at different angles relative to another group of one or more slots 20. The distribution and/or configuration of slots 20 can also include, to the extent applicable, any of those disclosed in U.S. Pat. Publication No. US 2004/0181174, the entire disclosure of which is herein incorporated by reference.

Slots 20 may be provided to enhance the flexibility of tubular member 18 while still allowing for suitable torque transmission characteristics. Slots 20 may be formed such that one or more rings and/or tube segments interconnected by one or more segments and/or beams that are formed in tubular member 18, and such tube segments and beams may include portions of tubular member 18 that remain after slots 20 are formed in the body of tubular member 18. Such an interconnected structure may act to maintain a relatively high degree of torsional stiffness, while maintaining a desired level of lateral flexibility. In some embodiments, some adjacent slots 20 can be formed such that they include portions that overlap with each other about the circumference of tubular member 18. In other embodiments, some adjacent slots 20 can be disposed such that they do not necessarily overlap with each other, but are disposed in a pattern that provides the desired degree of lateral flexibility.

Additionally, slots 20 can be arranged along the length of, or about the circumference of, tubular member 18 to achieve desired properties. For example, adjacent slots 20, or groups of slots 20, can be arranged in a symmetrical pattern, such as being disposed essentially equally on opposite sides about the circumference of tubular member 18, or can be rotated by an angle relative to each other about the axis of tubular member 18. Additionally, adjacent slots 20, or groups of slots 20, may be equally spaced along the length of tubular member 18, or can be arranged in an increasing or decreasing density pattern, or can be arranged in a non-symmetric or irregular pattern. Other characteristics, such as slot size, slot shape, and/or slot angle with respect to the longitudinal axis of tubular member 18, can also be varied along the length of tubular member 18 in order to vary the flexibility or other properties. In other embodiments, moreover, it is contemplated that the portions of the tubular member, such as a proximal section, or a distal section, or the entire tubular member 18, may not include any such slots 20.

As suggested herein, slots 20 may be formed in groups of two, three, four, five, or more slots 20, which may be located at substantially the same location along the axis of tubular member 18. Alternatively, a single slot 20 may be disposed at some or all of these locations. Within the groups of slots 20, there may be included slots 20 that are equal in size (i.e., span the same circumferential distance around tubular member 18). In some of these as well as other embodiments, at least some slots 20 in a group are unequal in size (i.e., span a different circumferential distance around tubular member 18). Longitudinally adjacent groups of slots 20 may have the same or different configurations. For example, some embodiments of tubular member 18 include slots 20 that are equal in size in a first group and then unequally sized in an adjacent group. It can be appreciated that in groups that have two slots 20 that are equal in size and are symmetrically disposed around the tube circumference, the centroid of the pair of beams (i.e., the portion of tubular member 18 remaining after slots 20 are formed therein) is coincident with the central axis of tubular member 18. Conversely, in groups that have two slots 20 that are unequal in size and whose centroids are directly opposed on the tube circumference, the centroid of the pair of beams can be offset from the central axis of tubular member 18. Some embodiments of tubular member 18 include only slot groups with centroids that are coincident with the central axis of the tubular member 18, only slot groups with centroids that are offset from the central axis of tubular member 18, or slot groups with centroids that are coincident with the central axis of tubular member 18 in a first group and offset from the central axis of tubular member 18 in another group. The amount of offset may vary depending on the depth (or length) of slots 20 and can include other suitable distances.

Slots 20 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the tubular member 18 is formed by cutting and/or removing portions of the tube to form slots 20. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing catheter 10 may include forming slots 20 in tubular member 18 using these or other manufacturing steps.

In at least some embodiments, slots 20 may be formed in tubular member using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow tubular member 18 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form tubular member 18 without being limited by a minimum cutting blade size. Consequently, tubular members 18 may be fabricated for use in neurological devices or other devices where a relatively small size may be desired.

The materials that can be used for the various components of catheter 10 (and/or other catheters disclosed herein) and the various tubular members disclosed herein may include those commonly associated with medical devices. For simplicity purposes, the following discussion makes reference to tubular member 18 and other components of catheter 10. However, this is not intended to limit the devices and methods described herein, as the discussion may be applied to other similar tubular members and/or components of tubular members or devices disclosed herein.

Tubular member 18, core wire 16, and/or other components of catheter 10 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of core wire 16 and/or tubular member 18 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of catheter 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of catheter 10 to achieve the same result.

In some embodiments, a degree of Magnetic Resonance Imaging (MRI) compatibility is imparted into catheter 10. For example, to enhance compatibility with MRI machines, it may be desirable to make core wire 16 and/or tubular member 18, or other portions of the catheter 10, in a manner that would impart a degree of MRI compatibility. For example, core wire 16 and/or tubular member 18, or portions thereof, may be made of a material that does not substantially distort the image and create substantial artifacts (i.e., gaps in the image). Certain ferromagnetic materials, for example, may not be suitable because they may create artifacts in an MRI image. Core wire 16 and/or tubular member 18, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Referring now to core wire 16, the entire core wire 16 can be made of the same material along its length, or in some embodiments, can include portions or sections made of different materials. In some embodiments, the material used to construct core wire 16 is chosen to impart varying flexibility and stiffness characteristics to different portions of core wire 16. For example, a proximal section and a distal section of core wire 16 may be formed of different materials, for example, materials having different moduli of elasticity, resulting in a difference in flexibility. In some embodiments, the material used to construct the proximal section can be relatively stiff for pushability and torqueability, and the material used to construct the distal section can be relatively flexible by comparison for better lateral trackability and steerability. For example, the proximal section can be formed of straightened 304v stainless steel wire or ribbon and the distal section can be formed of a straightened super elastic or linear elastic alloy, for example a nickel-titanium alloy wire or ribbon.

In embodiments where different portions of core wire 16 are made of different materials, the different portions can be connected using a suitable connecting technique and/or with a connector. For example, the different portions of core wire 16 can be connected using welding (including laser welding), soldering, brazing, adhesive, or the like, or combinations thereof. These techniques can be utilized regardless of whether or not a connector is utilized. The connector may include a structure generally suitable for connecting portions of a guidewire, portions of a core wire, or the like. One example of a suitable structure includes a structure such as a hypotube or a coiled wire which has an inside diameter sized appropriately to receive and connect to the ends of the proximal portion and the distal portion. Other suitable configurations and/or structures can be utilized for the connector including those connectors described in U.S. Pat. Nos. 6,918,882 and 7,071,197 and/or in U.S. Patent Pub. No. 2006-0122537, the entire disclosures of which are herein incorporated by reference.

A sheath or covering (not shown) may be disposed over portions or all of catheter shaft 12 that may define a generally smooth outer surface for catheter 10. In other embodiments, however, such a sheath or covering may be absent from a portion of all of catheter 10. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the catheter 10 may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portions of device 10. Alternatively, the sheath may comprise a lubricious, hydrophilic, protective, or other type of coating. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A fixed wire balloon catheter, comprising:
a core wire;
an inner tubular member attached to the core wire;
wherein the inner tubular member has a plurality of slots formed therein;
a catheter shaft disposed about the inner tubular member;
wherein an inflation lumen is defined between the catheter shaft and the core wire;
a balloon coupled to the catheter shaft, the balloon having a distal portion; and
a crossing tip coupled to the distal portion of the balloon.

2. The fixed wire balloon catheter of claim 1, wherein the inner tubular member has a longitudinal axis and wherein at least some of the slots formed in the inner tubular member are disposed at an angle relative to the longitudinal axis.

3. The fixed wire balloon catheter of claim 1, wherein the inner tubular member has a longitudinal axis and wherein at least some of the slots formed in the inner tubular member lie within a plane that is normal to the longitudinal axis.

4. The fixed wire balloon catheter of claim 1, wherein the balloon has a distal end and wherein a distal end of the inner tubular member extends distally beyond the distal end of the balloon.

5. The fixed wire balloon catheter of claim 1, wherein the balloon has a distal end and wherein a distal end of the inner member is disposed at or proximally of the distal end of the balloon.

6. The fixed wire balloon catheter of claim 1, wherein the balloon has a distal end and wherein a distal end of the core wire extends distally beyond the distal end of the balloon.

7. The fixed wire balloon catheter of claim 1, wherein the inner tubular member has a distal end and wherein a distal end of the core wire extends distally beyond the distal end of the inner tubular member.

8. The fixed wire balloon catheter of claim 1, wherein the crossing tip has an uneven outer surface.

9. The fixed wire balloon catheter of claim 1, wherein the crossing tip has a groove formed therein.

10. The fixed wire balloon catheter of claim 9, wherein the groove is a helical groove.

11. The fixed wire balloon catheter of claim 1, wherein the crossing tip has a thread extending radially outward therefrom.

12. The fixed wire balloon catheter of claim 1, wherein the balloon has a distal waist and wherein the crossing tip is attached to the distal waist.

13. The fixed wire balloon catheter of claim 1, wherein the balloon is a drug eluting balloon.

14. The fixed wire balloon catheter of claim 1, wherein an inner sleeve is disposed along an inner surface of the inner tubular member and wherein the inner sleeve forms a fluid tight seal that prevents fluid from passing through at least some of the slots.

15. The fixed wire balloon catheter of claim 1, wherein an outer sleeve is disposed along an outer surface of the inner tubular member and wherein the outer sleeve forms a fluid tight seal that prevents fluid from passing through at least some of the slots.

16. A fixed wire balloon catheter, comprising:
a core member;
a tubular member disposed about the core member, the tubular member having a distal portion and having an inflation lumen defined therein;
wherein the tubular member has a plurality of slots formed therein;
a balloon coupled to the distal portion of the tubular member, the balloon having a distal waist;
a sealing member coupled to the tubular member, the sealing member contacting at least one of an inner surface and an outer surface of the tubular member;
wherein the sealing member forms a fluid tight seal that prevents fluid from passing through at least some of the slots formed in the tubular member; and
a crossing tip coupled to the distal waist of the balloon.

17. The fixed wire balloon catheter of claim 15, wherein the core member has a first length, wherein the tubular member has a second length, and wherein the first length is at least as long as the second length.

18. The fixed wire balloon catheter of claim 15, wherein the tubular member extends distally beyond the balloon.

19. The fixed wire balloon catheter of claim 15, wherein the tip member has a helical groove formed therein, a helical thread disposed thereabout, or both.

20. A fixed wire balloon catheter, comprising:
a catheter shaft having a length;
a balloon coupled to the catheter shaft;
a torque assembly extending through the catheter shaft, the torque assembly comprising:
a core member,
a tubular member attached to the core member, the tubular member having a plurality of slots formed therein, and
a crossing tip attached to the tubular member;
wherein the torque assembly has a length that is at least as long as the length of the catheter shaft; and
wherein the torque assembly is rotatable relative to the catheter shaft.
